(19) United States
(12) Patent Application Publication
(10) Pub. No.: US 2014/0031617 A1
(43) Pub. Date: Jan. 30, 2014

(54) PENIS EXTENSION DEVICE

(71) Applicant: Joseph Maini, Worcester, MA (US)

(72) Inventor: Joseph Maini, Worcester, MA (US)

(21) Appl. No.: 13/934,878

(22) Filed: Jul. 3, 2013

Related U.S. Application Data
(60) Provisional application No. 61/676,172, filed on Jul. 26, 2012.

(51) Int. Cl. A61F 5/41

(52) U.S. Cl. CPC A61F 5/41
USPC 600/38

(57) ABSTRACT

An extension device with a tubular body having the shape of a penis. The tubular body may include a rounded sidewall, a top end and a bottom end. The tubular body may have a substantially hollow core. An entrance at the bottom end may lead into the hollow core. The tubular body of the present invention may further include openings on the sidewalls that expose the hollow core.

### CROSS-REFERENCE TO RELATED APPLICATION

This application claims the benefit of priority of U.S. provisional application No. 61/676,172, filed Jul. 26, 2012, the contents of which are herein incorporated by reference.

### BACKGROUND OF THE INVENTION

The present invention relates to a penis extension device and, more particularly, to a penis extension device with openings allowing for the user to experience increased pleasure.

Currently, penis extension devices may be used to increase pleasure for a woman during intercourse. However, penis extensions tend to be thick and constrict the penis. Therefore, the pleasure experienced by the male wearing the penis extension may be greatly reduced. Further, the extensions are difficult to keep discrete and are very cumbersome.

As can be seen, there is a need for a discrete penis extension device that facilitates increased pleasure for the wearer.

### SUMMARY OF THE INVENTION

In one aspect of the present invention, a penis extension device comprises: a penis shaped tubular body comprising a rounded sidewall, a top end, and a bottom end, wherein the tubular body has a substantially hollow core forming an entrance comprising a penis opening at the bottom end; wherein the tubular body comprises at least one opening at least partially formed around a circumference of the rounded sidewall, exposing the substantially hollow core.

These and other features, aspects and advantages of the present invention will become better understood with reference to the following drawings, description and claims.

### BRIEF DESCRIPTION OF THE DRAWINGS

FIG. 1 is a perspective of the present invention stored in a receptacle;

FIG. 2 is a perspective view of the present invention outside of the receptacle of FIG. 1 in a fully extended position;

FIG. 3 is a bottom view of the present invention stored in the receptacle of FIG. 1;

FIG. 4 is a section view of the present invention, taken along line 4-4 of FIG. 2;

FIG. 5 is a section view of the present invention, taken along line 5-5 of FIG. 1;

FIG. 6 is a perspective view of the present invention, illustrated in a semi-extended position; and

FIG. 7 is a perspective view of the present invention, illustrated in a compressed position configured to be placed within the receptacle of FIG. 1.

### DETAILED DESCRIPTION OF THE INVENTION

The following detailed description is of the best currently contemplated modes of carrying out exemplary embodiments of the invention. The description is not to be taken in a limiting sense, but is made merely for the purpose of illustrating the general principles of the invention, since the scope of the invention is best defined by the appended claims.

Broadly, an embodiment of the present invention provides an extension device with a tubular body having the shape of a penis. The tubular body may include a rounded sidewall, a top end and a bottom end. The tubular body may have a substantially hollow core. An entrance at the bottom end may lead into the hollow core. The tubular body of the present invention may further include openings on the sidewalls that expose the hollow core.

The present invention may offer men a more discrete method of adding girth and length to their penis. The present invention may expose the frenulum of the man's penis and thereby allow sensation to be experienced by the male. Further, a condom may be used with the prosthesis to prevent sexually transmitted disease from spreading. The penis extension may be modified to include different colors, textures, lubrication, artificial piercings, vibrators, various types of condoms, and the like.

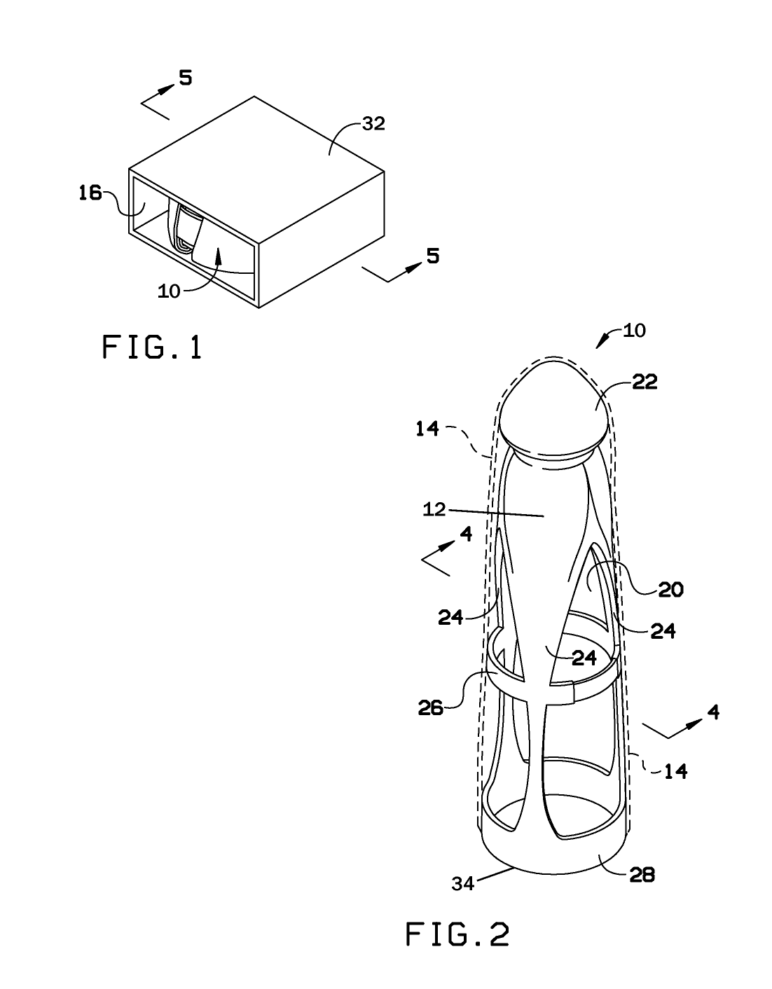

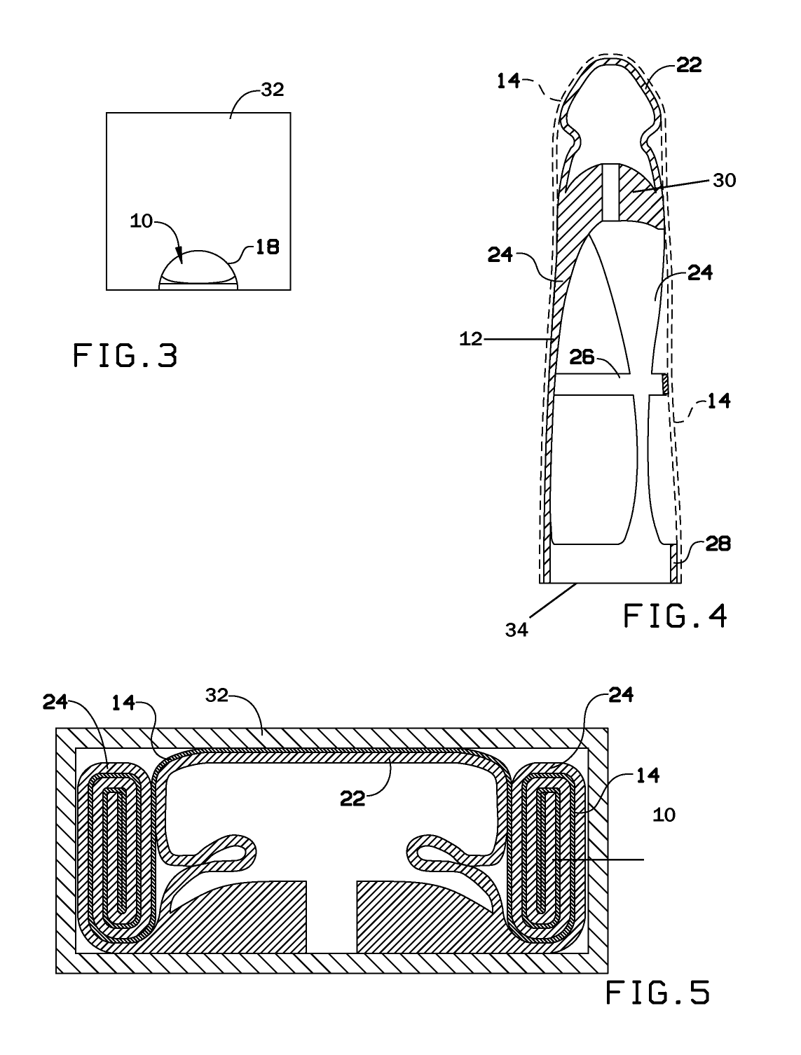

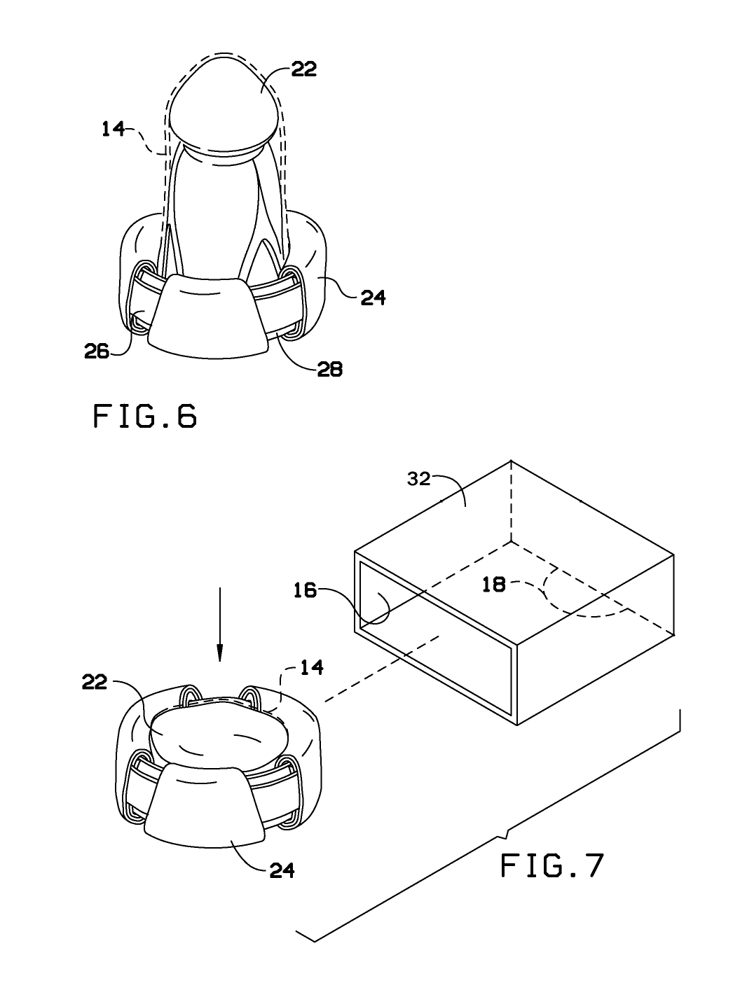

Referring to FIG. 1 through 7, the present invention may include a penis extension 10 made of a polymer. The penis extension 10 may include a tubular body 12 that is shaped like a penis. The tubular body 12 has a rounded sidewall, a top end and a bottom end. The tubular body 12 may have a substantially hollow core forming a rim and thereby forming an opening at the bottom end. The opening at the bottom end may be an entrance 34 to the substantially hollow core and may receive the penis of the user. The tubular body 12 of the present invention may include at least one opening 20 at least partially formed around the circumference of the rounded sidewall. The opening 20 in the sidewall exposes the substantially hollow core.

In certain embodiments, the tubular body 12 may include a base ring 28, a center ring 26 and a plurality of side straps 24. The base ring 28 may be at the bottom end of the tubular body 12 and may include the entrance 34 to the substantially hollow core. The center ring 26 may be in between the base ring 38 and the top end of the tubular body 12. The plurality of side straps 24 may run along the length of the tubular body 12, from the base ring 38 and through the center ring 26. The plurality of side straps 24 may connect together near the top end of the tubular body 12. The openings 20 may be formed in between the plurality of side straps 24. In certain embodiments, there may be three side straps 24.

In certain embodiments, the present invention may include a hollow head portion 22 at the top end of the tubular body 12. The hollow head portion 22 may be divided from the substantially hollow core by a support divider 30. The support divider 30 may be used to further add support and firmness to the tubular body 12. In certain embodiments, the support divider 30 may include an opening that connects the substantially hollow core with the hollow head portion 22.

The present invention may be in a compressed state and an expanded state. As illustrated in FIG. 2, the expanded state may include the tubular body 12 being fully extended. The tubular body 12 may be converted from the expanded state to the compressed state by folding the tubular body 12 upward upon itself from the bottom end to the top end, as illustrated in FIG. 5. In certain embodiments, the present invention may further include a receptacle 32 with a receptacle opening 16 and a finger slot 18. The receptacle 32 may further include more than one piece hinged together. Therefore, a user may place the tubular body 12 in the receptacle 32 when the tubular body 12 is in the compressed state, as illustrated in FIG. 7. The penis extension 10 may thereby be easily carried and transported allowing the user to be more discrete.

In certain embodiments, a condom 14 may be surrounding the surface of the penis extension 10. The penis extension 10 and condom 14 may work together in the extraction and placement process. The penis extension 10 may add length and girth, while leaving the frenulum open for sensation. The condom 14 may offer STD and pregnancy protection. A specialized condom may be created with thin spots over the frenulum to add pleasure while wearing the condom 14. The receptacle 32 may allow the penis extension 10 and condom 14 to be compressed for easy storage and dispensing.

To make the present invention a negative mold would need to be placed in a traditional penis extension mold to create the open areas for the frenulum. Once completed, a condom may be placed over the penis extension and rolled up upon itself as to make a single roll. This completed assembly may then be compressed and slid into the receptacle and then placed in a packaging machine for wrapper.

To use the present invention a user may: open the packaging and remove the receptacle containing the penis extension and condom; remove the condom and penis extension from the receptacle by sliding a finger in the provided finger slide; allow a few seconds for the prosthesis to expand to its extended size; align the open area to leave sensation for frenulum, with an alignment tool that may be provided; slide on to the penis similar to a condom; and after use, the device may be easily removed from the flaccid penis by simply pulling on the tip.

It should be understood, of course, that the foregoing relates to exemplary embodiments of the invention and that modifications may be made without departing from the spirit and scope of the invention as set forth in the following claims.

What is claimed is:

1. A penis extension device comprising:
a penis shaped tubular body comprising a rounded sidewall, a top end, and a bottom end, wherein the tubular body has a substantially hollow core forming an entrance comprising a penis opening at the bottom end;
wherein the tubular body comprises at least one opening at least partially formed around a circumference of the rounded sidewall, exposing the substantially hollow core.

2. The penis extension of claim 1, wherein the tubular body comprises:
a base ring at the bottom end comprising the entrance of the tubular body;
a center ring in between the base ring and the top end of the tubular body; and
a plurality of side straps running lengthwise of the tubular body from the base ring through the center ring and connecting together near the top end of the tubular body, wherein the at least one opening is formed in between the plurality of side straps.

3. The penis extension device of claim 1, further comprising an expanded state and a compressed state, wherein the expanded state comprises the tubular body fully extended, and the compressed state comprises the tubular body folded upward upon itself from the bottom end to the top end.

4. The penis extension device of claim 3, further comprising a receptacle comprising a receptacle opening, wherein the penis extension device is stored within the receptacle in the compressed state.

5. The penis extension device of claim 1, further comprising a condom surrounding an outside surface of the tubular body.

6. The penis extension device of claim 1, wherein the top end comprises a substantially hollow head portion.

7. The penis extension device of claim 7, further comprising a support divider separating the hollow head portion from the substantially hollow core.

8. The penis extension device of claim 7, wherein the support divider comprising an opening connecting the substantially hollow core and the hollow head portion.
